(19) United States
(12) Patent Application Publication
(10) Pub. No.: US 2005/0182023 A1
(43) Pub. Date: Aug. 18, 2005

(54) Process to prepare semicarbazones' and/or tiosemicarbazones' formulations using cyclodextrins and their derivatives and products obtained by this process

(76) Inventors: Ruben Dario Sinisterra Millan, Minas Gerais (BR); Marcio M. Coelho, Minas Gerais (BR); Rafael Pinto Vieira, Belo Horizonte-Minas Gerais (BR); Leticia Regina de Souza Teixeira, Belo Horizonte-Minas Gerais (BR); Maria Carolina Doreto, Vespasiano-Minas Gerais (BR); Heloisa de Oliveira Beraldo, Belo Horizonte-Minas Gerais (BR)

Correspondence Address: NIXON & VANDERHYE, PC, 901 NORTH GLEBE ROAD, 11TH FLOOR, ARLINGTON, VA, 22203, US

(21) Appl. No.: 10/503,735

(22) PCT Filed: Feb. 5, 2003
(86) PCT No.: PCT/BR03/00018

(30) Foreign Application Priority Data
Feb. 6, 2002 (BR) PI0200751.7

(51) Int. Cl. A61K 31/724; C08B 37/16

(52) U.S. Cl. 514/58; 536/46

(57) ABSTRACT

The preparation of semicarbazone and/or thiosemicarbazone formulations with cyclodextrins and their derivatives and products obtained by this process. The invention is characterized by obtaining inclusion compounds of semicarbazone and/or thiosemicarbazones with cyclodextrins and their derivatives, which were tested in experimental epilepsy models and allowed the reduction of the anticonvulsant dose from 100 mg/kg. This means an improvement in the bioavailability of the compounds in biological systems. These results obtained in animal models make semicarbazones and/or thiosemicarbazones included in cyclodextrins and their derivatives new anticonvulsant candidates. The invention is also characterized by the improved efficacy of semicarbazones and/or thiosemicarbazones included in cyclodextrins and their derivatives in comparison to free components. In addition the present invention is also characterized by the pain killer effect of semicarbazones and thiosemicarbazones. The invention is also characterized by a lowering of the dose necessary for the pain killer effect of semicarbazones and thiosemicarbazones upon inclusion into cyclodextrins.

The present invention is characterized by the preparation of semicarbazone and/or thiosemicarbazone formulations using cyclodextrins and their derivatives and products obtained by this process.

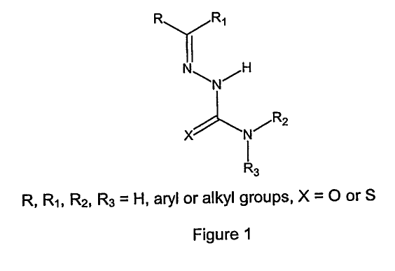

Thiosemicarbazones (FIG. 1, Generic structure of semicarbazones and/or thiosemicarbazones) are compounds with a large range of biological applications, presenting antitumoral, antiviral, antibacterial, antimalarial, antituberculosis, fungicide, anti-HIV and anticonvulsant activities [West, D. X.; Padhyé, S. B.; Sonawane, P. B., Structure and Bonding, 76, 1 (1991); Dimmock, J. R., Pandeya, S. N., Quail, J. W., Pugazhenthi, U., Allen, T. M., Kao, G. Y., Balzarini, J., DeClercp, E., Eur. J. Med. Chem., 30 (1995)].

Semicarbazones (FIG. 1) are analogues of the above mentioned compounds in which oxygen substitutes sulfur. A series of publications reports on the anticonvulsant activity of semicarbazones [Dimmock, J. R., Pandeya, S. N., Quail, J. W., Pugazhenthi, U., Allen, T. M., Kao, G. Y., Balzarini, J., DeClercp, E., Eur. J. Med. Chem., 30, (1995); Dimmock, J. R., Sidhu, K. K., Thayer, R. S., and cols. J. Med. Chem., 36 (1993); Dimmock, J. R., Puthucode, R. N. Smith, J. M. e cols., J. Med. Chem., 39 (1996)]. In particular, compounds derived from arylsemicarbazones present anticonvulsant activity in the central nervous system [Kadaba, P. K.; Lin, Z.; U.S. Pat. No. 5,942,527 (1999); Dimmock, J. R.; Puthucode, R. N.; WO9640628, MX9709311, JP11506109, U.S. Pat. No. 5,741,818 (1997); Fujibayashi, Y.; Yokoyama, A.; U.S. Pat. No. 5,843,400 (1996)].

Structural variations can lead to significant modifications of the biological activity of semicarbazones and thiosemicarbazones, and the literature contains studies on structure-activity relationships [West, D. X.; Padhyé, S. B.; Sonawane, P. B., Structure and Bonding, 76, 1 (1991); Kadaba, P. K.; Lin, Z.; U.S. Pat. No. 5,942,527 (1999)].

Semicarbazones are stable, can be orally administered [Kadaba, P. K.; Lin, Z.; U.S. Pat. No. 5,942,527 (1999)] and proved to be more active as anticonvulsants than phenytoin and phenobarbital, which are the most used drugs in neurologic clinic to treat epilepsies in humans [Dimmock, J. R., WO9406758 (1994)]. Additionally, they present none or very low toxicity [Dimmock, J. R.; Puthucode, R. N., WO9640628, MX9709311, JP11506109, U.S. Pat. No. 5,741,818 (1997); Fujibayashi, Y.; Yokoyama, A., U.S. Pat. No. 5,843,400 (1996)].

In the state of art, it is observed that semicarbazones and thiosemicarbazones present anticonvulsant activity in two experimental models of epilepsy: the subcutaneous pentylenetetrazole screen, and the maximum electroshock screen [Dimmock, J. R.; Sidhu, K. K.; Thayer, R. S.; Mack, P.; Duffy, M. J.; Reid, R. S.; Quail, J. W.; Pugazhenthi, U.; Ong, A.; Bikker, J. A.; Weaver, D. F., J. of Med. Chem., 36, 16 (1993); Dimmock, J. R.; Pandeya, S. N.; Quail, J. W.; Pugazhenthi, U.; Allen, T. M.; Kao, G. Y.; Balzarini, J.; DeClercq, E., Eur. J. Med. Chem., 30, 303 (1995); Dimmock, J. R.; Sidhu, K. K.; Tumber, S. D.; Basran, S. K.; Chen, M.; Quail, J. W.; Yang, J.; Rozas, I.; Weaver, D. F., Eur. J. Med. Chem., 30, 287 (1995); Dimmock, J. R.; Puthucode, R. N.; Smith, J. M.; Heltherington, M.; Quail, W. J.; Pughazenti, U.; Leshler, T.; Stables, J. P., J. Med. Chem., 39, 3984 (1996); Dimmock, J. R.; Vashishtha, S. C.; Stables, J. P., Eur. J. Med. Chem., 35, 241 (2000); Kadaba, P. K.; Lin, Z., U.S. Pat. No. 5,942,527 (1999); Dimmock, J. R.; Puthucode, R. N., WO9640628, MX9709311, JP11506109, U.S. Pat. No. 5,741,818 (1997); Fujibayashi, Y.; Yokoyama, A., U.S. Pat. No. 5,843,400 (1996)].

The existing patents that report the anticonvulsant activity of semicarbazones and thiosemicarbazones are described below.

U.S. Pat. No. 5,942,527 Kadaba et al. (1999), prepared new pharmaceutical formulations containing hydrazones, hydrazines, thiosemicarbazones and semicarbazones and tested the anticonvulsant activity of these compounds in rats with electroshock induced seizures. The compounds showed to be active in oral administrations in doses of 100 mg/Kg and presented low neurotoxicity.

U.S. Pat. No. 5,741,818 (1997), (MX9709311, WO9640628, AU9659938, FI9704447, NO9705663, EP836591, CZ9703874, NZ309707, HU9802637, JP11506109, BR9609408, AU715897, KR99022408) Dimmock et al., prepared semicarbazones derived from 4-phenoxy or 4-phenylthio-benzaldehyde and tested the anticonvulsant activity of these compounds in rats with electroshock induced seizures. The compounds presented no neurotoxicity in doses up to 500 mg/Kg.

WO9406758 (1996) Dimmock, prepared aryl semicarbazones and tested their effect on the central nervous system as anticonvulsants and in the prevention of epileptic seizures. These compounds showed to be more active than phenytoin and phenobarbital in vivo, and than the corresponding semicarbazides. They are stable, can be given orally, and present low or no neurotoxicity.

Epilepsy is a morbid condition known for over 3,000 years. Due to its incidence and its dramatic manifestations, and its social impact, it has attracted the attention of scholars and laymen.

The World Health Organization (WHO) defines epilepsy as a chronic cerebral disorder with varied etiology characterized by recurring seizures caused by excessive cerebral neuronal discharge. To the present, the pathogenesis of the cerebral disorder is unknown.

The incidence is estimated at about 50 and 120 out of 100,000 people. About 3-5% of the general population will experiment one or more seizures sometime in life [Cockerell, O. C.; Shorvon, S. D.; Epilepsia: Conceitos atuais, Current Medical Literature Ltd. Lemos Editorial e gráficos Ltda. SP (1997)]. There are several frequent types of epilepsy in the population, occurring at any age and sex, most often starting in childhood or adolescence.

Epileptic seizures are clinic events which reflect either a temporary dysfunction of a small part of the brain (focal seizures) or of a larger area involving the two cerebral hemispheres (generalized seizures).

Epilepsies with identifiable causes (symptomatic) occur in only 30% of the cases and are associated to several disturbs, including infections, traumas, brain tumors, cerebral vascular disease and Alzheimer-Pick disease. Idiopathic epilepsies are transmitted genetically and manifest in certain age groups, and cryptogenic epilepsies are those presumed to have an organic basis, but with unclear etiology. Eur. J. Med. Chem., 30, 287 (1995); Dimmock, J. R.; Puthucode, R. N.; Smith, J. M.; Heltherington, M.; Quail, W. J.; Pughazenti, U.; Leshler, T.; Stables, J. P., J. Med. Chem., 39, 3984 (1996); Dimmock, J. R.; Vashishtha, S. C.; Stables, J. P., Eur. J. Med. Chem., 35, 241 (2000); Kadaba, P. K.; Lin, Z., U.S. Pat. No. 5,942,527 (1999); Dimmock, J. R.; Puthucode, R. N., WO9640628, MX9709311, JP11506109, U.S. Pat. No. 5,741,818 (1997); Fujibayashi, Y.; Yokoyama, A., U.S. Pat. No. 5,843,400 (1996)].

The existing patents that report the anticonvulsant activity of semicarbazones and thiosemicarbazones are described below.

U.S. Pat. No. 5,942,527 Kadaba et al. (1999), prepared new pharmaceutical formulations containing hydrazones, hydrazines, thiosemicarbazones and semicarbazones and tested the anticonvulsant activity of these compounds in rats with electroshock induced seizures. The compounds showed to be active in oral administrations in doses of 100 mg/Kg and presented low neurotoxicity.

U.S. Pat. No. 5,741,818 (1997), (MX9709311, WO9640628, AU9659938, FI9704447, NO9705663, EP836591, CZ9703874, NZ309707, HU9802637, JP11506109, BR9609408, AU715897, KR99022408) Dimmock et al., prepared semicarbazones derived from 4-phenoxy or 4-phenylthio-benzaldehyde and tested the anticonvulsant activity of these compounds in rats with electroshock induced seizures. The compounds presented no neurotoxicity in doses up to 500 mg/Kg.

WO9406758 (1996) Dimmock, prepared aryl semicarbazones and tested their effect on the central nervous system as anticonvulsants and in the prevention of epileptic seizures. These compounds showed to be more active than phenytoin and phenobarbital in vivo, and than the corresponding semicarbazides. They are stable, can be given orally, and present low or no neurotoxicity.

Epilepsy is a morbid condition known for over 3,000 years. Due to its incidence and its dramatic manifestations, and its social impact, it has attracted the attention of scholars and laymen.

The World Health Organization (WHO) defines epilepsy as a chronic cerebral disorder with varied etiology characterized by recurring seizures caused by excessive cerebral neuronal discharge. To the present, the pathogenesis of the cerebral disorder is unknown.

The incidence is estimated at about 50 and 120 out of 100,000 people. About 3-5% of the general population will experiment one or more seizures sometime in life [Cockerell, O. C.; Shorvon, S. D.; Epilepsia: Conceitos atuais, Current Medical Literature Ltd. Lemos Editorial e gráficos Ltda. SP (1997)]. There are several frequent types of epilepsy in the population, occurring at any age and sex, most often starting in childhood or adolescence.

Epileptic seizures are clinic events which reflect either a temporary dysfunction of a small part of the brain (focal seizures) or of a larger area involving the two cerebral hemispheres (generalized seizures).

Epilepsies with identifiable causes (symptomatic) occur in only 30% of the cases and are associated to several disturbs, including infections, traumas, brain tumors, cerebral vascular disease and Alzheimer-Pick disease. Idiopathic epilepsies are transmitted genetically and manifest in certain age groups, and cryptogenic epilepsies are those presumed to have an organic basis, but with unclear etiology.

Epileptic seizures are those which occur under epileptic conditions and are characterized by motor shaking of some parts of the body (partial seizures) or all the body (generalized seizures).

Non-epileptic convulsive seizures are common symptoms of acute neurologic diseases such as meningitis, cranium encephalic traumas, cerebral vascular diseases and others. Metabolic changes may also be associated to convulsive seizures. Non-organic seizures are those without any pathologic anatomic change correlated to the disturb. Non-organic seizures are most commonly psychogenic (conversion hysterias).

Hyperexcitability and synchronism seem to be essential characteristics of the cerebral substrates that can generate a set of neural (neurochemical, neuroanatomic, electrophysiologic, etc.) and behavioral changes [Moraes, M. F. D.; Epilepsia Experimental: estudos eletrofisiológicos e comportamentais em modelos animais de crises convulsivas audiogênicas, Doctorate thesis presented at Faculdade de Medicina de Ribeirão Preto of Universidade de São Paulo (1998)] that characterize convulsive seizures.

To the present, it has not been possible to establish a simple and practical classification of epilepsies, i.e., of the several chronic diseases whose main symptom is represented by recurring seizures. In contrast, the classification of the different types of convulsive seizures is relatively easy [Goodman and Gilman's, The Pharmacological Basis of Therapeutics, 9′ ed., Pergamon Press, New York (1996)]. The classification of epilepsies is based on criteria relative to convulsive seizures, such as frequency, triggering factors, clinical condition, physiopathologic mechanisms, etiology and the age seizures start.

Generalized epileptic seizures are those which occur with loss of conscience and which can either present generalized, bilateral and symmetric motor changes, and vegetative disturbs or not. Absence seizure is generalized and does not have motor manifestation. The responsible neuronal discharge may appear in any area of the brain and may spread to other regions, even involving both cerebral hemispheres.

Among the generalized epileptic seizures distinguishes a convulsing group (tonic-clonic, tonic, clonic, infant spasms, and bilateral myoclonus), and a non-convulsing group (typical absences or petit mal seizures, atypical absences, atonic seizures and akinetic seizures.).

Focal or partial epileptic seizures are those in which electroencephalographic changes are restricted, at least in the beginning, to a specific region of the encephalon. These seizures are classified based on their clinical characteristics as: motor seizures (Jacksonianas, masticatory), sensitive (somatosensitive, cardiocirculatory, respiratory), psychic seizures (delusions, hallucinations) and psychomotor seizures (automatisms).

Treatment is symptomatic, since the drugs available inhibit seizures and there is neither effective prophylaxis nor cure. Keeping to the drug posology is important due to the need of long term treatment with the ensuing side effects of many drugs.

The ideal anticonvulsant drug would suppress all seizures without bringing on any side effects. However, the presently used drugs not only control the convulsant activity in some patients, but also often produce side effects of variable degree, from minimal changes of the CNS to death by aplastic anemia or hepatic insufficiency. It is possible to achieve complete control of seizures in 50% of the patients, and another 25% may improve significantly. Most success is achieved with newly diagnosed patients and it depends on factors such as the type of convulsion, family history, and extent of associated neurological changes [Goodman and Gilman's, The Pharmacological Basis of Therapeutics, 9′ ed., Pergamon Press, New York (1996).]

Drugs effective against the most common forms of epileptic convulsions, partial tonic-clonic, and secondarily generalized seizures, seem to result from one of two mechanism. One mechanism reduces the repetitive discharge maintained by a neuron, an effect mediated by the promotion of the inactivity of Na⁺ channels activated by voltage. Another mechanism seems to involve the potentialization of the synaptic inhibition mediated by the γ-aminobutyric acid (GABA), and an intermediate effect through the pre-synaptic action of some drugs and the post-synaptic action of others. The most efficient drugs against a less common form of epileptic convulsion, the absence seizure, lead to the reduction of the activity of the Ca²⁺ channel activated by special voltage, known as T current.

Phenobarbital was the first organic agent synthesized and acknowledged as having anticonvulsant activity. Its sedative properties led investigators to test and demonstrate its efficacy in suppressing convulsive seizures. In a historic discovery, Merrit and Putnam (1938) [Merrit, H. H.; Putnam, T. J.; Arch. Neurol. Psychiatry, 39, 1003 (1938)] developed the electroshock convulsive seizure screen in experimental animals to test the anticonvulsant efficacy of chemical agents. They found out from research with a variety of drugs that phenytoin suppressed convulsions without a sedative effect. The electroshock convulsive seizure test is extremely valuable since the drugs efficient against the tonic extension of the hinter legs induced by electroshock are generally effective against partial and tonic-clonic convulsions in humans. Another classification test, induction of convulsive seizures by subcutaneous pentylenetetrazol (sc-PTZ) is useful to identify drugs efficient against absence seizures in humans. Before 1965, the chemical structures of many drugs were rather similar to that of Phenobarbital. These drugs include hydantoins, oxazolydinadiones and succinimides. The agents introduced after 1965 were benzodiazepines (clonazepam and clorazepate), iminostilben (carbamazepine), a carboxylic acid (valproic acid), a phenyltriazine (lamotrigine), and a cyclic analogue to GABA (gabapentin.) [Goodman and Gilman's, The Pharmacological Basis of Therapeutics, 9^{th} ed., Pergamon Press, New York (1996)].

Phenytoin is efficient against all types or partial and tonic-clonic convulsions, but not absence seizures. It is the most extensively studied anticonvulsant agent both in laboratory and in clinical practice. Phenytoin exerts its anticonvulsant action without causing generalized depression of the CNS. In toxic doses, it can provoke excitation signals and a type of decerebration rigidity in lethal levels. The most significant effect of phenytoin is its capacity to change the pattern of convulsions caused by maximum electroshock. It is possible to eliminate the characteristic tonic phase completely, however, the residual clonic convulsion can be heightened and prolonged. This modifying action of the convulsion seizure is also observed for other drugs that are efficient against generalized tonic-clonic convulsions. In contrast, phenytoin does not inhibit clonic convulsions induced by pentylenotetrazole. Intravenous administration of phenytoin inhibits convulsion seizures in a susceptible model.

The anticonvulsant use of carbamazepine was approved in the United States in 1974, having being used since the 60's to treat trigeminal nerve neuralgia. It is presently considered a first line drug in the treatment of partial and tonic-clonic convulsions.

The use of valproic acid was approved in the USA in 1978, after being used for over a decade in Europe. The anticonvulsant properties of valproate were discovered serendipitously when it was used as a vehicle for other compounds that were being investigated against convulsions. The valproic acid (n-dipropylacetic acid) is a simple branched chain carboxylic acid.

To study the mechanisms and the physiological consequences of epilepsy and also the action of anticonvulsant mechanisms, experimental chronic or acute models were used. The models most used for this purpose are the genetic, maximum and minimum electroshock, and chemical ones.

In the electroshock model, the epileptic seizures are induced by electric currents from electrodes placed on the head of an animal. Browning (1995) [Browning, R. A., Anatomy of generalized convulsive seizures, in Idiopathic generalized epilepsies. Clinical, experimental and genetic aspects, A. Malafosse, P et al (Eds.), John Libbey & Company Ltd. (1994)]. The literature reports that depending on the cerebral region where the current is applied, different types of seizures can be obtained. With trans-auricular electrodes, it was possible to obtain generalized tonic-clonic seizure and with trans-corneal electrodes, limbic seizures.

In genetic models, two combined factors are necessary to obtain a seizure. First, a specific genetic predisposition whose origin is in an anomaly in the neurotransmitters associated with the cholinergic, catecholaminergic, serotoninergic systems and/or amino acids [Jobe, P. C.; Laird, H. E.; Biochem. Pharmacol, 30, 3137 (1981)]. The second factor, also called trigger, includes environmental stimuli such as intermittent light, sound, hyperthermia, postural changes and/or new circumstances. Endogenous neurochemical alterations or a hormonal unbalance can also work as triggers. Therefore, for the onset of an epileptic seizure in the genetic model, an inborn predisposition is necessary to seizure together with one or more either exogenous or endogenous triggers. An individual may never have a seizure due to the lack of predisposition or trigger(s) [Aicardi, J.; Course and prognosis of certain childhood epilepsies with predominantly myoclonic seizures and Wada, J. A.; Penry, J. K. and cols; Advances. in epileptology, The X^{th} Epilepsy International Symposium. New York; Ravem, 159 (1980)].

Audiogenic epilepsy in rats is a genetic model in which seizures are induced by high intensity acoustic stimuli. Four rat colonies with this characteristic were selected. A line derived from Wistar, called. WAR-Wistar Audiogenic Rats was bred in Brazil at the laboratory of Neurophysiology and Experimental Neurology of the Physiology department of Faculdade de Medicina de Ribeirão Preto, Universidade de São Paulo [Garcia-Cairasco, N.; Doretto, M. C.; Lobo, R. B., Epilepsia, 31, 815 (1990)]. A breed of this line is kept at the breeding facilities of Departmento de Fisiologia e Biofisica of Instituto de Ciências Biológicas, Universidade Federal de Minas Gerais, Belo Horizonte [Doretto, M. C.; Oliveira-e-Silva, M.; Ferreira, M.; Garcia-Cairasco, N.; Reis, A. M., Proceedings Congresso Latinoamericano de Epilepsia, Santiago, Chile (2000)]. In WARs, seizures are characterized by running, jumping, atonic falls, tonic convulsions, partial and generalized tonic-clonic convulsions, and clonic spasms [Garcia-Cairasco, N.; Sabbatini, R. M. E., Braz. J. Me. Biol. Res., 16, 171 (1983); Garcia-Cairasco, N.; Doretto, M. C.; Prado, P.; Jorge, B. P. D.; Terra, V. C.; Oliveira, J. A. C., Behav. Brain Res., 58, 57 (1992)].

A drug can be chemically modified to alter its properties such as biodistribution, pharmacokinetics and solubility. Several methods have been used to increase drug solubility and stability, including organic solvents, emulsions, liposomes, pH adjustments, chemical modifications and complexations of drugs with appropriate encapsulating agents such as cyclodextrins.

Cyclodextrins are cyclic oligosaccharides with six, seven or eight glucopyranose units. Due to steric interactions, cyclodextrins form a cyclic structure shaped like a truncated cone with an apolar internal cavity. They are chemically stable compounds which can be regioselectively modified. Cyclodextrins (hosts) form complexes with several hydrophobic molecules (guests), including guest molecules either completely or partially into the cavity. Cyclodextrins have been used to solubilize and encapsulate drugs, perfumes and flavors as described in the literature [Szejtli, J., Chemical Reviews, 98, 1743 (1998); Szejtli, J., J. Mater. Chem., 7, 575 (1997)]. In respect to detailed toxicity, mutagenicity, teratogenicity and carcinogenicity studies, cyclodextrins present low toxicity [Rajewski, R. A.; Stella, V.; J. Pharmaceutical Sciences, 85, 1142 (1996)], particularly hydroxylpropyl-β-cyclodextrin [Szejtli, J. Cyclodextrins: Properties and applications. Drug Investig., 2(suppl. 4):11 (1990)]. Except for some cyclodextrin derivatives which provoke damage to erythrocytes in high concentrations, these products in general are not hazardous. The use of cyclodextrins as food additives has been authorized in countries like Japan and Hungary, and for more specific uses in France, and Denmark. In addition, they are obtained from a renewable source from starch degradation. All these characteristics are added reasons for the discovery of new applications. The molecular structure of cyclodextrins is a truncated cone with approximate Cₙ symmetry. The primary hydroxyls are located on the narrow side of the cone, and the secondary hydroxyls on the broad side. Despite the stability due to the intramolecular hydrogen bonds, it is flexible enough to allow considerable shape modifications.

Cyclodextrins are moderately soluble in water, methanol, and ethanol, and readily soluble in aprotic apolar solvents such as dimethyl sulfoxide, dimethylformamide, N,N-dimethylacetamide and pyridine.

There are many works in state-of-art on the effects of the increase in solubility of low soluble guests through inclusion into cyclodextrins. The physical-chemical characteristics and stability of inclusion compounds are well described. [Szejtli, J., Chemical Reviews, 98, 1743 (1998); Szejtli, J., J. Mater. Chem., 7, 575 (1997)].

The development of new pharmaceutical formulations tends to modify the present concept of drug in the short term. Thus, recently several systems were developed to administer drugs with the purpose of modeling release kinetics, improving drug absorption and stability, or targeting them to specific cellular populations. As a result appear polymeric compositions, cyclodextrins, liposomes, emulsions, multiple emulsions which serve as carriers of active principles. These formulations can be administered via intramuscular, intravenous, or subcutaneous injection, orally, inhalation, or with implanted or injected devices.

The present invention is characterized by obtaining inclusion compounds of semicarbazones and/or thiosemicarbazones in cyclodextrins and their derivatives which once tested in experimental models allowed the reduction of anticonvulsant dose from 100 mg/kg to 35 mg/kg. This means an increase in bioavailability of compounds in biological systems. Hence inclusion compounds between semicarbazones and/or thiosemicarbazones and cyclodextrins and their derivatives could be new candidates as anticonvulsant agents.

The present invention is also characterized by the increase in the efficacy of the inclusion compounds cyclodextrins-semicarbazones and/or thiosemicarbazones and their derivatives in comparison to free components.

The present invention reports for the first time “the pain killer effect of semicarbazones and thiosemicarbazones”.

The present invention is also characterized by a lowering of the dose necessary for the pain killer effect of semicarbazones and thiosemicarbazones upon inclusion into cyclodextrins.

In addition this technology is also characterized by the preparation of the formulations of inclusion compounds of semicarbazones and thiosemicarbazones into cyclodextrinsc and semicarbazones and thiosemicarbazones, using biodegradable polymers, lipossomes, emulsion and multiple emulsion or combinations thereof.

The present invention can be better understood through the following non-limiting examples.

### EXAMPLE 1

### Preparation of Inclusion Compound Between hydroxypropyl-β-cyclodextrin and Semicarbazone Using for Example Benzaldehyde Semicarbazone

Preparation of drug/CD solid complex. Benzaldehyde semicarbazone (BS) was obtained as described in the literature. The inclusion compound (IC) with hidroxypropyl-β-cyclodextrin (HP-β-CD) was prepared by mixing BS and HP-β-CD in water in 1:1 molar ratio with stirring for 24 hours. The suspension was submitted to a freeze-drying process (Labconco Freezone model 177) during 48 hours. The inclusion grade was determined by UV spectroscopy using a HP8453 diode array spectrometer. The absorbance was measured at 282 nm in methanol, using a 1 cm path length quartz cell. The calibration curve was made using known concentrations of BS in methanol. A physical mixture of the same BS: HP-β-CD molar ratio was obtained for comparison.

Infrared Studies

The first evidence for the host-guest interaction was obtained from the modification of the infrared absorptions of BS and HP-β-CD upon inclusion. Table 1 lists the main absorptions in the infrared spectra of BS, HP-β-CD, the physical mixture (PM) and the inclusion compound (IC). In the spectrum of HP-β-CD the absorptions at 3425 cm-1, 2920 cm-1, 1650 cm-1 and 1030 cm-1 were attributed to ν(OH), ν(C—H), δ(O—H) and ν(C—O—C) respectively. In the spectrum of BS the absorptions at 3463 cm-1, 3339 cm-1 and 1600 cm-1 were attributed to ν(N—H), ν(NH2) and ν(C═N) respectively. The ν(C—H) bands of BS were observed in the 2900-3100 cm-1 range. Two absorptions attributed to ν(C═O) were found at 1690 and 1650 cm-1. In the spectrum of the PM the ν(N—H), ν(NH2) and ν(C—H) absorptions do not appear separately but lay underneath the ν(OH) envelope centered at 3400 cm-1. Also, the intensity of the ν(C═N) absorption and that of ν(C═O) at 1650 cm-1 decrease whereas the intensity of the ν(C═O) absorption at 1690 cm-1 remains practically unchanged, suggesting some hydrogen bonding between BS and HP-β-CD in the PM. The same absorption (broad) is observed at 3400 cm-1 in the inclusion compound and the intensities of the two ν(C═O) maxima as well as that of ν(C═N) undergo a substantial decrease with concomitant modification in the intensity ratio, indicating the formation of a new species.

Higher thermal stability was observed for BS after host-guest interaction. The TG curve of IC (FIG. 2) presents a plateau until 300° C. when decomposition occurs, as evidenced by its DTG curve (inset). The TG/DTG curves for HP-β-CD show a weight loss of 6.6% in the 33-122° C. range, associated to the release of five water molecules and reaches a plateau of stability until 350° C. when decomposition occurs. BS undergoes decomposition at 256° C. The TG/DTG curves of PM exhibit two decomposition peaks, associated to HP-β-CD and BS.

For HP-β-CD, DSC measurements show one endothermic peak at 52.7° C. corresponding to the release of water and two exothermic peaks at 310.2° C. and 371.4° C., corresponding to the decomposition of the molecule. The DSC curve of the inclusion compound exhibit one endothermic peak at 52.3° C. attributable to the release of water molecules. Interestingly, the fusion of BS is not observable indicating the interaction of BS and the CD cavity. Moreover, the DSC curve of PM shows approximately the same thermal behavior, suggesting that some inclusion is already observed.

The XRD powder pattern diffraction analyses gave further support for the formation of a supramolecular compound between BS and HP-β-CD. The BS XRD powder diffraction pattern shows sharp peaks, characteristic of a crystalline compound. In contrast, HP-β-CD is amorphous. The XRD pattern of PM and of the inclusion compound as compared to that of free HP-β-CD suggest the formation of a higher organized system upon inclusion or association.

NMR spectroscopy provided strong support for the formation of a host-guest complex between BS and HP-β-CD. In free BS the hydrogen relaxation times T1 for H1, H2, H2′ were determined in the 1.56-1.65 s-1 range and those for H3 and H3′ were 1.60 and 1.69 s-1 respectively. In addition, the measured T1 for H5, H6 and H7 were 0.93, 0.83 and 0.33 s-1 respectively. Upon inclusion, the values of T1 of H1, H2 and H2′ shifted to 1.38-1.42 s-1, T1 of H3 and H3′ to 1.40 and 1.46 s-1 respectively and T1 of H5, H6 and H7 to 0.83, 0.73 and 0.29 s-1 respectively (see FIG. 5). Upon host-guest interaction the values of hydrogen relaxation times T1 decrease suggesting greater rigidity of the guest's hydrogens [15]. This effect is more pronounced for the aromatic hydrogens, indicating recognition of the phenyl moiety by the CD cavity. The variations observed in T1 for the semicarbazone moiety could be due to hydrogen bonding between the semicarbazone hydrogens and the hydoxyl groups of the hydroxypropyl substituent on the cyclodextrin.

The signals in the spectra of BS and HP-β-CD are in agreement with data reported in the literature.

Upon inclusion, all hydrogen and carbon signals shift to lower frequencies in agreement with recognition of the phenyl group by the CD cavity, as suggested by the T1 measurements. Interestingly, the resonance signals of the semicarbazone moiety of BS are also affected ie. NH2 (Δ=0.126), N—H (Δ=0.084), C—H (Δ=0.063) and C═O (Δ=0.238), probably due hydrogen bonding to the hydroxyl groups of the HP-β-CD.

### EXAMPLE 2

### Preparation of Drug/CD Solid Complex-BS was Obtained as Described in the Literature

The inclusion compound with β-cyclodextrin (β-CD) was prepared by mixing BS and β-CD in water in 1:1 molar ratio with stirring for 48 hours. The suspension was submitted to a freeze-drying process (Labconco Freezone model 177) during 72 hours. A 1:1 BS:β-CD physical mixture was obtained for comparison. The 1:1 BS:β-CD molar ratio in the inclusion compound was confirmed by the Higuchi and Connors method, 11 measuring the BS absorbance at 280 nm in water with a 1 cm path length quartz cell.

As in the case of the inclusion compound BS/HP-β-CD, the first evidence for host-guest interaction was obtained from the modification of the infrared absorptions of BS and β-CD upon inclusion. In the FTIR spectrum of β-CD the absorptions at 3400 cm-1,2925 cm-1, 1640 cm-1 and 1025 cm-1 were attributed to ν(OH), ν(C—H), δ(O—H) and ν(C—O—C) respectively. 12 In the spectrum of BS the absorptions at 3463 cm-1, 3395 cm-1 and 1600 cm-1 were attributed to ν(N—H), ν(NH2) and ν(C═N) respectively. The ν(C—H) bands of BS were observed in the 2900-3100 cm-1 range. Two absorptions attributed to ν(C═O) were found at 1690 and 1650 cm-1.

Comparison between the FTIR spectra of BS, the BS/β-CD inclusion compound and the physical mixture reveal important changes upon inclusion. The BS ν(N—H) and ν(NH2) bands at 3463 cm-1, and 3395 cm-1 respectively were also observed in the spectrum of the physical mixture and in that of the inclusion compound. However, a narrowing of the β-CD absorptions was observed in the inclusion compound, probably due to the breaking of hydrogen bonds upon host-guest interaction. Besides, the intensities of ν(C═O) at 1690 cm-1 and ν(C═N) at 1600 cm-1 of BS undergo a substantial decrease in the spectrum of the inclusion compound which is not observed in the spectrum of the physical mixture, indicating molecular recognition of BS by the β-CD cavity. Crystal structure determinations of BS showed that the distance between the carbonyl carbon and the center of the aryl ring is 9.5 Å. 14 On the other hand it is well established that the length distance of β-CD is 7.9 Å, 15 indicating that the cavity could accommodate the aromatic ring as well as part of the BS semicarbazone moiety.

The TG/DTG and DSC curves for β-CD and BS present thermal behaviors as related in the literature.

The TG/DTG curves of the physical mixture exhibit thermal profiles associated to β-CD and BS. The DSC curve shows four endothermic peaks at 70.6° C., 214.7° C., 306.3° C. and 326.3° C., corresponding to β-CD and BS thermal phenomena. The last two peaks, attributed to melting and caramelization of β-CD are observed separately, in contrast to the DSC curve of β-CD, which shows only one thermal event.

The thermal behavior of the BS/β-CD inclusion compound is entirely different. Its TG curve presents a weight loss in the 30-80° C. range attributed to the release of water molecules followed by a second loss in the 190-250° C. range, corresponding to the BS melting. Decomposition occurs at 360° C., as evidenced by the DTG curve. The DSC curve of the BS/β-CD inclusion compound exhibits one endothermic event at 58.7° C., but the strong peak at 78.3° C. and 70.6° C. originally observable in the β-CD and in the physical mixture curves respectively is now absent, indicating the release of water molecules upon inclusion. In addition, the peak at 208.8° C. corresponds to the BS melting and finally that at 332.8° C. can be associated to a new thermal phenomenon of the supramolecular compound. Interestingly, the DSC curves of the BS/β-CD and BS/HP-β-CD inclusion compounds are very similar.

The XRD powder pattern diffraction analyses gave further support for the formation of a supramolecular compound between BS and β-CD. The XRD powder diffraction patterns of BS and β-CD exhibit sharp peaks, characteristic of crystalline compounds. The XRD pattern of the physical mixture shows peaks characteristic of BS and β-CD. In contrast, the BS/β-CD inclusion compound presents a pattern that suggests a loss of crystallinity with formation of a less organized system upon inclusion. Comparison of the XRD patterns of the BS/β-CD inclusion compound with that of the BS/HP-β-CD analogue, prepared previously, indicates that the latter is more amorphous and consequently more water soluble.

The signals in the spectra of BS and β-CD were in agreement with data reported in the literature. Upon host-guest interaction, all hydrogen signals of BS shift to lower frequencies (data not shown) and the carbon signals to higher frequencies. Interestingly, the Cl, CH and C═O signals exhibit the most significant shifts upon interaction, confirming the inclusion of the BS molecule from the aryl ring to the carbonyl oxygen of the semicarbazone moiety into the β-CD cavity as ascertained by infrared data.

Changes were observed in all relaxation times but the most significant variations were obtained for the ring Hydrogens, followed by N—H and C—H, in accordance with the 13C NMR and infrared results. It is worth noting that the minor T1 change was observed for the NH2 hydrogens, suggesting that this group is less affected by host-guest interaction, probably due to its longer distance from the hydrophobic aryl ring and consequently from the β-CD cavity.

### EXAMPLE 3

Animals. Wistar rats from the main breeding stock of the Institute of Biological Sciences, Federal University of Minas Gerais, Brazil, and Wistar Audiogenic Rats (WARs) from our own inbred colony, maintained at the animal facilities of the Physiology Department, weighing 250-300 g, were used. They were kept at 24° C., in groups of 5 per cage receiving chow pellets and water ad libitum. The light/dark cycle was 12 h: 12 h, with lights on at 7:00 am and lights off at 7:00 pm. Efforts were made in order to avoid any unnecessary distress to the animals, in accordance to the Guidelines for Animal Experimentation of Federal University of Minas Gerais, Brazil.

Induction and evaluation of audiogenic seizures (AS). Sound stimulus (120 dB) was delivered into an acoustic chamber through a loud speaker, until tonic seizures appeared, or during a maximum of 1 minute. Behavior was evaluated by a severity index (SI) ranging from SI=0.0 to SI=1.0 (maximum) as described elsewhere.

Typically WARs present running fits, jumping and atonic falling followed by tonic-clonic seizures and clonic spasms (SI≧0.85). Animals were stimulated three times, once every three days before the beginning of experiments, in order to screen them for seizure severity (control recording). Seven days after the third stimulation they were used in the experiments.

Tests were conducted always after 4:00 pm and animals were used in the experiments at least one week after the last screening stimulus. All susceptible animals used in these experiments displayed SI≧0.85 at the beginning of the studies (at least generalized tonic-clonic seizures). To evaluate the effect of decreasing on AS severity, it was taken as criteria the blockade of the tonic component of seizure, which means to obtain SI<0.61.

Induction and evaluation of maximum electroshock-induced seizures (MES). Electroshock seizures were induced by electric stimulus, produced by an ELEKTROSCHOCKGERÄT apparatus (Karl Kolbe, Scientific Technical Supplies, Frankfurt, Germany) using a current of 70 mA, 60 Hz, during 1 second through a pair of ear clip electrodes.

The behavioral evaluation was carried out by evaluating the tonic component in a four points scale as follows: 0=no seizure; 1=forelimb extension without hind limb extension; 2=complete forelimb extension and partial hind limb extension; 3=complete hind limb extension, which stays parallel to the tail. To evaluate the effect of decreasing on electroshock induced seizures severity, it was taken as criteria the blockade of complete fore- and hind limb extension (score≦1).

BS and the IC, administered by intraperitoneal route (ip) and by gavage (vo), were tested in the two experimental models of generalized tonic-clonic seizures: the maximal electroshock-induced seizures (MES) and the audiogenic seizures (AS) models.

Comparison of the anticonvulsant effect of free benzaldehyde semicarbazone (BS) and the HP-β-CD/BS inclusion compound in the maximum electroshock screening (MES). In the MES model, BS blocked the hindlimb extension in about 90% of the animals (males) at 100 mg/Kg/ip and vo as observed in the literature [5]. The IC blocked completely the hindlimb extension at 35 mg/Kg/ip and vo in 100% of the animals and at 25 mg/Kg/ip in 67% of the animals (FIG. 6). Moreover, the IC at 50 and 100 mg/Kg, ip and vo, in addition to the seizures blockage, caused behavioral disturbances, characterized by a decreased motor activity and responsiveness to environmental stimuli. Rats were examined 30 and 240 minutes after administration of the IC (vo). Whereas free BS exhibits no activity after 240 minutes [5], the IC blocked hindlimb extension in 60% of the animals, indicating a slow release of the drug (FIG. 7).

Comparison of the anticonvulsant effect of free benzaldehyde semicarbazone (BS) and the HP-β-CD/BS inclusion compound in the audiogenic seizures (AS). In the AS model, BS blocked the tonic component of seizures in 33, 50 and 83% of the animals at 50, 75 and 100 mg/Kg/ip respectively (FIG. 8). The IC at 100 mg/Kg, in addition to the seizures blockage, caused behavioral disturbances similar to those observed in the MES tests. At 35 mg/Kg (vo and ip), the IC blocked the tonic component of seizures in 100% of the male animals and 60% of the female animals, without the undesirable effects previously described (FIG. 9). In this model the IC exhibits no activity 240 minutes after administration.

In conclusion in the MES model of epilepsy the minimum dose necessary to produce anticonvulsant activity decreased from 100 mg/Kg for the free semicarbazone to 35 mg/Kg/vo and 25 mg/Kg/ip for the inclusion compound, which represents 65-75% of dose reduction. Moreover, upon inclusion a slow release of the drug was observed. In the AS model the minimum dose necessary to produce anticonvulsant activity decreased from 100 mg/Kg (vo and ip) for the free semicarbazone to 35 mg/Kg for the inclusion compound, which represents 65% of dose reduction. These results suggest that the host-guest strategy could be used in the preparation of new pharmaceutical formulations of anticonvulsant drugs.

### EXAMPLE 4

In the MES model of epilepsy the minimum dose necessary to produce anticonvulsant activity decreased from 100 mg/Kg (ip or vo) for the free semicarbazone to 25 mg/Kg/vo (75%) and 15 mg/Kg/ip (85%) for the BS/β-CD inclusion compound. Comparison with the results obtained previously by us for the BS/HP-β-CD inclusion compound, which allowed dose reduction of 75% ip (see FIG. 5) and 65% vo9 reveals that the host-guest strategy that uses β-CD is even more effective. The reasons for this difference could be either the lower water solubility of the BS/β-CD inclusion compound as compared to the BS/HP-β-CD analogue or the β-CD greater adhesion to the mucous wall, 20 which would allow a more sustained release.

In conclusion, taking into consideration that currently used drugs cause significant side effects which may limit their maximal usefulness, the new strategy could be successfully employed in the preparation of pharmaceutical formulations of anticonvulsants.

### EXAMPLE 5

Pain Killer Effect of Semicarbazones, Thiosemicarbazones and Their Inclusion Compounds in Cyclodextrins

The effect of the inclusion compound betacyclodextrin-benzaldehyde semicarbazone (CBS) or hydroxypropil-betacyclodextrin-benzaldehyde semicarbazone (HP-CBS) on the nociceptive response induced by formaldehyde in mice was investigated. Subcutaneous injection of formaldehyde 0.92% in the right hindlimb induced a nociceptive behaviour characterised by paw licking. Previous intraperitoneal injection of CBS (35 mg/kg), but not of HP-CBS (35 mg/kg), inhibited the second phase of the nociceptive response induced by formaldehyde. Both compounds changed neither the motility of the animals in the open-field test nor the time spent in the rota-rod, suggesting that the antinociceptive effect does not result from motor incoordination, muscle relaxing effect or an nonspecific depression of the central nervous system.

1. Process of preparation of formulations of semicarbazones and/or thiosemicarbazones with cyclodextrins and their derivatives and products obtained by this process, characterized by the mixture of organo-aqueous solutions of cyclodextrins or cyclodextrin derivatives selected from the group containing alkyl, hydroxyalkyl, hydroxypropyl and acyl or cross-linked cyclodextrins or cyclodextrin polymers with organo-aqueous solutions of semicarbazones and/or thiosemicarbazones.

2. Preparation of formulations of semicarbazones and/or thiosemicarbazones with cyclodextrins and their derivatives, in accordance with claim 1, characterized by the increase in water solubility of semicarbazones and/or thiosemicarbazones.

3. Preparation of formulations of semicarbazones and/or thiosemicarbazones with cyclodextrins and their derivatives, in accordance with claim 1, characterized by the reduction of the therapeutic dose from 100 mg/kg to 25 mg/kg in the electroshock model and in rats with audiogenic epileptic susceptibility (WAR).

4. Preparation of formulations of semicarbazones and/or thiosemicarbazones with cyclodextrins and their derivatives, in agreement with claim 1, characterized by the increase in bioavailability and efficacy of semicarbazones and/or thiosemicarbazones.

5. Process to prepare the formulations of metallic complexes of semicarbazones and/or thiosemicarbazones with cyclodextrins and their derivatives and products obtained by this process, characterized by the mixture of organo-aqueous solutions of cyclodextrins or their derivatives selected from the group containing alkyl, hydroxyalkyl, hydroxypropyl and acyl or cross-linked cyclodextrins or cyclodextrin polymers with organo-aqueous solutions of metallic complexes of semicarbazones and/or thiosemicarbazones.

6. Process of preparation of formulations of metallic complexes of semicarbazones and/or thiosemicarbazones with cyclodextrins and their derivatives, in agreement with claim 5, characterized by the increase in water solubility.

7. Product of semicarbazones and/or thiosemicarbazones with cyclodextrins and their derivatives, in agreement with claim 1, characterized by the increase in bioavailability and efficiency of semicarbazones and/or thiosemicarbazones.

8. Product of semicarbazones and/or thiosemicarbazones with cyclodextrins and their derivatives, in agreement wit claim 1, characterized by the reduction of the therapeutic dose from 100 mg/kg to 25 mg/kg in electroshock model and rats with audiogenic epileptic susceptibility (WAR).

9. Product of semicarbazones and/or thiosemicarbazones and their metallic complexes with cyclodextrins and their derivatives, in agreement with claim 5, characterized by the formation of inclusion compounds between cyclodextrins and their derivatives and metallic complexes of semicarbazones and/or thiosemicarbazones.

10. Process of preparation of formulations of semicarbazones and/or thiosemicarbazones characterized by a pain killer effect.

11. Preparation of formulations of semicarbazones and/or thiosemicarbazones with cyclodextrins and their derivatives, in accordance with claim 1, characterized by a pain killer effect.
